(19) United States
(12) Patent Application Publication
(10) Pub. No.: US 2006/0275798 A1
(43) Pub. Date: Dec. 7, 2006

(54) Detection of organisms using a media sachet and primer directed nucleic acid amplification

(76) Inventors: John C. Steichen, Landenberg, PA (US); George Tice, Penns Grove, NJ (US); Beckyjo A. Smith, Hudson, WI (US)

Correspondence Address: PATENT GROUP;ATTN: J. KENNETH JOUNG, DLA PIPER RUDNICK GRAY CARY US LLP, 203 N. LASALLE ST., SUITE 1900, CHICAGO, IL, 60601, US

(21) Appl. No.: 11/397,825

(22) Filed: Apr. 4, 2006

Related U.S. Application Data
(60) Provisional application No. 60/668,020, filed on Apr. 4, 2005.
(60) Provisional application No. 60/708,037, filed on Aug. 12, 2005.

(51) Int. Cl. C12Q 1/68; C12P 19/34

(52) U.S. Cl. 435/6; 435/91.2

(57) ABSTRACT

A method of detecting a target organism in a sample mixture comprising preparing at least one complex sample mixture, performing primer directed nucleic acid amplification of the complex sample mixture; and examining the primer directed nucleic acid amplification result to detect for presence or absence of amplification product of the target organism, wherein the presence or absence of the amplification product is indicative of the presence or absence of the target organism in the sample mixture. Preparing the complex sample mixture comprises adding to a container at least one sachet containing at least a nutrient concentrate and a sample mixture suspected of containing the target organism.

### CROSS-REFERENCE TO RELATED APPLICATIONS

This application claims the benefit of U.S. Provisional Application No. 60/668,020, filed Apr. 4, 2005 and U.S. Provisional Application No. 60/708,037, filed Aug. 12, 2005, each of which is hereby incorporated by reference in its entirety.

### FIELD OF THE INVENTION

The field of the invention relates to molecular biology and particularly to methods and compositions for preparing samples and detecting target organisms contained therein.

### BACKGROUND

Nucleic acid based methods, and in particular polymerase chain reaction (PCR) methods, are powerful analytical tools in the detection and identification of organisms such as bacteria. Nucleic acid based methods include the amplification of any desired specific nucleic acid sequence contained in a nucleic acid or mixture thereof. The use of these methods for bacterial detection in known in the art. However, these methods typically demand adherence to strict protocols under strict conditions and often require personnel of advanced skills and training in order to achieve a reliable result.

In all nucleic acid based methods for detection of organisms, and particularly in a PCR-based test procedure, extraneous components that may enhance or inhibit the test reaction make it difficult to obtain credible results. These extraneous components are often present in testing of food-derived matrices for bacterial contaminants that effect quality, such as pathogens, spoilage, and off-taste promoters and the like.

In order to increase the effectiveness of nucleic acid based methods, such as PCR, it is desirable to increase to measurable levels the organism concentration in the sample to be tested. This is often accomplished by mixing a sample to be tested with a nutrient medium that enables the growth of the organism population in the sample.

Media preparation and sterilization are well known to those skilled in the art. Large batches of media and additives can be prepared, and smaller portions then transferred, for example, into homogenizer medium bags for carrying out sample enrichment. However, media preparation at each testing laboratory is often expensive, labor intensive, and subject to error, especially during the measuring and transferring operations. The food and environmental testing industry in particular considers this process of media preparation, sterilization, storage, and introduction, to be burdensome. The availability of liquid media solutions, even in flexible medium bags, is still often undesirable, due to shipment drawbacks arising from the weight and volume of the solutions. Also, a wide variety of analyses needed in the food and environmental testing industry may require many different media in terms of dilution, volume, nutrient profile and/or other factors.

### SUMMARY OF THE INVENTION

The present invention includes a method of detecting a target organism in a sample mixture comprising:

(a) preparing at least one complex sample mixture (preferably an enriched complex sample mixture), said preparing step comprising adding to a container (preferably a sterile container):
(i) at least one sachet containing at least a nutrient concentrate; and
(ii) a sample mixture (preferably a food sample mixture) suspected of containing a target organism;

(b) performing primer directed nucleic acid amplification (preferably PCR amplification, ligase chain reaction or strand-displacement amplification) of the at least one complex sample mixture; and

(c) examining the primer directed nucleic acid amplification result of step (b) to detect for presence or absence of amplification product of the target organism, wherein the presence or absence of the amplification product is indicative of the presence or absence of the target organism in the sample mixture.

### DETAILED DESCRIPTION OF PREFERRED EMBODIMENTS

The disclosure of each reference set forth herein is hereby incorporated by reference in its entirety.

As used herein, the term “PCR” means the Polymerase Chain Reaction as described by Mullis et al. in U.S. Pat. No. 4,683,195 and Mullis in U.S. Pat. No. 4,683,202.

As used herein, the term “complex sample mixture” refers to a mixture which may include one or more of any target organisms and non-target organisms, and organic materials and inorganic materials that will support the growth of a variety of organisms. The complex sample mixtures of the invention include, for example, organic growth-supporting substances, such as food matter, biological tissues, organic waste products, and the like.

As used herein, the term “target organism” refers to the organism from which the target nucleic acid is amplified. Target organisms may be members of defined mixed cultures, or exist as contaminants in complex matrices. Target organisms of particular interest are food-borne pathogens.

As used herein, the term “non-target organism” will be used interchangeably with the term “background organism” and will refer to any organism that is found in the presence of the target organisms but are not the target organisms. Non-target organisms may or may not be related genetically or biochemically to the target organisms. Those non-target organisms of most interest in the context of the present application are non-pathogenic food-borne organisms.

As used herein, the terms “non-selective growth” or “non-selective enrichment” refer to the growth of target and non-target organisms in a medium designed to resuscitate both target and non-target organisms which have been injured or compromised by the sample processing. “Non-selective growth media” will refer to either a liquid or solid media designed to encourage the growth of both target and non-target organisms. The non-selective growth media of the present invention may be buffered to allow for the variations in pH of a variety of different food matrices (e.g., due to bacteria producing acid during the enrichment process).

As used herein, the term “nucleic acid” refers to a molecule which can be single-stranded or double-stranded, comprising monomers (nucleotides) containing a sugar, phosphate and either a purine or pyrimidine. In bacteria, lower eukaryotes, and in higher animals and plants, “deoxyribonucleic acid” (DNA) refers to the genetic material while “ribonucleic acid” (RNA) is involved in the translation of the information from DNA into proteins.

As used herein, the term “total target organism nucleic acid” refers to any nucleic acid contained within the target organism that contains a distinctive sequence by which the target organism may be identified. Total target organism nucleic acid may include genomic DNA, RNA, episomal or plasmid DNA, or cDNA derived from genomic DNA or RNA.

As used herein, the term “target nucleic acid” refers to a nucleic acid fragment that is detected by the present detection method and is indicative of the presence of a target organism. The target nucleic acid is typically a unique portion of the genome of the target organism and specifically distinguishes the target organism from all other organisms.

As used herein, the term “amplification primer” or simply “primer” refers to a nucleic acid fragment or sequence that is complementary to at least one section along a strand of the target nucleic acid, wherein the purpose of the primer is to sponsor and direct nucleic acid replication of a portion of the target nucleic acid along that strand. Primers can be designed to be complementary to specific segments of a targeted sequence. In PCR, for example, each primer is used in combination with another primer forming a “primer set” or “primer pair”; this pair flanks the targeted sequence to be amplified. The term “primer”, as such, is used generally to encompass any sequence-binding oligonucleotide which functions to initiate the nucleic acid replication process.

As used herein, the term “control nucleic acid fragment” refers to a fragment of nucleic acid and may be bounded on both the 5′ and 3′ ends with either identical primer binding sites such that amplification of the control nucleic acid fragment may be accomplished with a single primer or with different primers. The control nucleic acid fragment will typically be of a size and base composition similar to the target nucleic acid to be detected. The control nucleic acid fragment may optionally reside as an insert in a plasmid or vector and may be incorporated into a tabletted reagent for the convenience of assay.

As used herein, the term “replication composition” or “nucleic acid replication composition” refers to a composition comprising ingredients for performing nucleic acid amplification. Nucleic acid replication compositions may be provided in a variety of forms including liquid mixtures as well as tabletted reagents. If PCR methodology is selected, the replication composition could include, for example, nucleotide triphosphates, at least one primer with appropriate sequences, DNA polymerase, suitable buffers and proteins. A “test replication composition” refers to a composition specifically designed to amplify a target nucleic acid.

As used herein, “positive control replication composition” refers to a composition that will amplify a control nucleic acid fragment.

As used herein, the term “tabletted reagent” will refer to a reagent useful for packaging the test and/or positive control replication compositions.

As used herein, the term “amplification product” refers to specific nucleic acid fragments generated from any primer-directed nucleic acid amplification reaction. Amplification products will generally be double stranded DNA (dsDNA) and will be amenable to being bound by intercalating agents.

As used herein, the term “primer directed nucleic acid amplification” or “primer-directed amplification” refers to any method known in the art wherein primers are used to sponsor replication of nucleic acid sequences in the linear or logarithmic amplification of nucleic acid molecules. Primer-directed amplification may be accomplished by any of several schemes known in this art, including but not limited to the polymerase chain reaction (PCR), ligase chain reaction (LCR) or strand-displacement amplification (SDA).

As used herein, the term “intercalating agent” means a fluorescent agent capable of intercalating into nucleic acid molecules. The term “intercalating agent” will be used interchangeably with the term “intercalating dye”. Intercalating agents emit a fluorescent signal when intercalated into the nucleic acid and will not generate any signal when not intercalated. Typical of intercalating agents are the cyanine dyes available from Molecular Probes, Inc. (Eugene, Oreg., USA).

As used herein, the term “Fluorescent Intensity Units” will be abbreviated “FIU”.

As used herein, the term “homogeneous detection” refers to a method for the detection of nucleic acid amplification products where no separation of products from template or primers is necessary.

In the food industry, food safety is of the utmost importance and therefore, simplified methods for testing food samples have become critical. The invention includes primer directed nucleic acid amplification (for example PCR-based methods) for the detection of a specific food-borne target bacteria in a complex sample mixture, where the method utilizes at least one sachet containing at least a nutrient concentrate. The PCR process is described in U.S. Pat. No. 4,683,195 (Mullis et al.) and U.S. Pat. No. 4,683,202 (Mullis et al.), each of which is hereby incorporated by reference in their entirety.

Generally in one aspect the invention includes a method of detecting a target organism in a sample mixture comprising:

(a) preparing at least one complex sample mixture (preferably an enriched complex sample mixture), said preparing step comprising adding to a container (preferably a sterile container):
(i) at least one sachet containing at least a nutrient concentrate; and
(ii) a sample mixture (preferably a food sample mixture) suspected of containing a target organism;

(b) performing primer directed nucleic acid amplification (preferably PCR amplification, ligase chain reaction or strand-displacement amplification) of the at least one complex sample mixture; and

(c) examining the primer directed nucleic acid amplification result of step (b) to detect for presence or absence of amplification product of the target organism, wherein the presence or absence of the amplification product is indicative of the presence or absence of the target organism in the sample mixture.

Optionally, between steps (a) and (b), additional sample preparation steps may be carried out, for example, separation of cells from the complex sample mixture, and/or cell lysis, and/or total nucleic acid extraction. Furthermore, regarding step (a)(i), the container may already contain water or other solution prior to the addition of the at least one sachet, or water or other solution may be provided to the container simultaneously with or after the addition of the at least one sachet.

The food sample mixture may contain a variety of components including non-target organisms and target organisms as well as other organic contaminants such as food debris. Typically, the target organisms include pathogenic bacteria commonly known to contaminate or infect food such as, for example, Salmonella, Listeria, E. coli, Campylobacter, Enterobacter sakazakii, Vibrio, and Clostridia. As is known in the art, the minimum industry standard for the detection of food-borne bacterial pathogens is a method that will reliably detect the presence of one pathogen cell in 25 g of food matrix.

Typically, prior to performing a primer directed nucleic acid amplification, at least one enriched complex sample mixture must be prepared so that the target bacteria can be grown or cultured and subsequently detected by biochemical, immunological or nucleic acid hybridization means. Non-selective growth may be performed in two stages: (1) homogenization of the nutrient and sample so they are intimately mixed, and (2) a longer period when the sample and nutrient are exposed to temperatures that foster growth of the target organism and/or non-target organisms. Optionally, during this second stage additional additives may be introduced into the nutrient medium to create a growth environment unfavorable to non-target organisms. These two stages are referred to as “sample enrichment” in the food industry.

Non-selective media have been developed for a variety of bacterial pathogens. One of ordinary skill in the art will know to select a medium appropriate for the particular organism to be enriched. A general discussion and recipes of non-selective media are described, for example, in Andrews et al., “Isolation and Identification of Salmonella Species,” Chapter 7 in Bacteriological Analytical Manual, 6th Edition, Association of Official Analytical Chemists, Arlington, Va. (1984).

After non-selective growth, a portion of the complex sample mixture is removed for further analysis. This may be accomplished by a variety of means, however, it is preferred if retrieval is done using a perforated piece of Porex™ high density polyethylene. Porex™ is particularly suited, as the small pore size of the polyethylene allows for maximum extraction of bacterial cells while excluding the large particles of food matrix. The retrieved, enriched bacterial sample portion is then lysed, for example in a lysis buffer, and subjected to a nucleic acid amplification protocol in the presence of an internal nucleic acid control.

Generally, in order to simplify sample enrichment, at least one sachet may be utilized, wherein the at least one sachet contains at least a nutrient concentrate. The at least one sachet of the invention may be used with any type of container, preferably sterilized, known in the art. Such containers suitable for use with the invention can include any medium bag known in the art including, but not limited to, a homogenizer bag such as the flexible culture medium bag described in the U.S. Provisional Patent Application No. 60/668,020, filed Apr. 4, 2005, which is hereby incorporated by reference in its entirety. The at least one sachet may be manufactured in any number of sizes and shapes, both of which are well known to those skilled in the art.

Typically, the at least one sachet (i.e., a small closed pouch) of the invention comprises a water-reactive material, wherein the water-reactive material dissolves, ruptures, disperses and/or disintegrates upon contact with water, so as to allow the nutrient concentrate or other component contained therein to be released into the water and form a liquid medium. Preferably, the material is water-soluble, such as a water-soluble polymeric material.

Alternatively, in place of the at least one sachet of the invention, there can be utilized a locus of containment comprising water-reactive polymers that include matrices or coatings of water-reactive material that enclose or envelop the nutrient concentrate solids so that they are not exposed to the air and adventitious organisms prior to the time of use.

The at least one sachet is preferably made from a water-soluble film. The at least one sachet may be made from two overlaid sheets of water-soluble film that are sealed together. Alternatively, the at least one sachet may comprise a sheet of water-soluble film sealed to a sheet of film that is not water-reactive. This alternative may be useful in preparing the at least one sachet with an extended tab for sealing between the sheets that form the medium bag, wherein the extended tab comprises the film that is not water-reactive. The at least one sachet may have a soluble seal that dissolves to release its nutrient concentrate or other contents.

The at least one sachet can be constructed of a material that is resistant to puncturing yet still water-reactive, and substantially transparent, such that a user can view and inspect the contents of the at least one sachet. In addition, the material is stable when dry and has a long shelf-life such that it may be stored for long periods of time without degrading.

The sheet(s) of polymeric film (i.e., the so-called “web stock”) used to prepare the at least one sachet may be produced using any combinations of the processes generally known in the art, such as monolayer or multilayer casting, blowing film, extrusion lamination, and adhesive lamination and combinations thereof.

Moreover, the at least one sachet is flexible such that the contents therein may be manipulated in order to enhance its dissolving or distribution properties. Still further, the at least one sachet can be pre-filled and/or pre-sterilized, particularly in the case of a dissolvable sachet.

The water-soluble film useful for the at least one sachet has a solubility in water of at least 50%, at least 75%, or even at least 95%. Solubility can be determined as follows. Fifty grams ±0.1 g of material is added to a 400-ml beaker of known weight, and 245 ml±1 ml of distilled water is added. This is stirred vigorously on magnetic stirrer set at 600 rpm for 30 minutes. Then, the mixture is filtered through a folded qualitative sintered-glass filter with the known pore sizes (typically less than 50 pm) to remove the insoluble material. The water is dried off from the collected filtrate by any conventional method, and the weight of the polymer residue is determined (which is the dissolved or dispersed fraction). Then, the % solubility or dispersability can be calculated.

The at least one sachet comprises preferably films or sheets of polymeric materials. The film or sheet can, for example, be obtained by casting, blow-molding, extrusion or blow extrusion of the polymer material, as known in the art. Preferred polymers, copolymers or derivatives thereof include one or more selected from polyvinyl alcohols, polyvinyl pyrrolidone, polyalkylene oxides, polyacrylamide, polyacrylic acid, cellulose, cellulose ethers, cellulose esters, cellulose amides, polyvinyl acetates, polycarboxylic acids and salts, polyaminoacids or peptides, polyamides, polyacrylamide, copolymers of maleic/acrylic acids, polysaccharides including starch and gelatine, natural gums such as xanthum and carragum. The polymer can also be polyacrylates and water soluble acrylate copolymers, methylcellulose, carboxymethylcellulose sodium, dextrin, ethylcellulose, hydroxyethyl cellulose, hydroxypropyl methylcellulose, maltodextrin, polymethacrylates, even more preferably polyvinyl alcohols, polyvinyl alcohol copolymers and hydroxypropyl methyl cellulose (HPMC). The polymer can have any weight average molecular weight, preferably from about 1000 to 1,000,000 or even from 10,000 to 300,000 or even from 15,000 to 200,000 or even from 20,000 to 150,000.

Mixtures of polymers can also be used. This may be beneficial to control the mechanical and/or dissolution properties of the sachet, depending on the application thereof and the required needs. For example, one polymer material has a higher water-solubility than another polymer material, and/or one polymer material has a higher mechanical strength than another polymer material. It may be preferred using a mixture of polymers, having different weight average molecular weights, for example a mixture of polyvinyl alcohol (PVA) or a copolymer thereof of a weight average molecular weight of 10,000-40,000, preferably around 20,000, and of PVA or copolymer thereof, with a weight average molecular weight of about 100,000 to 300,000, preferably around 150,000.

Also useful are polymer blend compositions, for example comprising a hydrolytically degradable and water-soluble polymer blend such as polylactide and polyvinyl alcohol, achieved by the mixing of polylactide and polyvinyl alcohol, typically comprising 1-35% by weight polylactide and approximately from 65% to 99% by weight polyvinyl alcohol, if the material is to be water-soluble.

The polymer can be present in the film from 60% to 98%, or 80% to 90%, hydrolyzed, to improve the dissolution of the material, and/or the levels of plasticizer, including water, in the film may be varied such that the dissolution is adjusted as required.

Also preferred is PVA film where the level of polymer in the film can be at least 60%. Such films can comprise a PVA polymer with similar properties to the film known under the trade reference M8630 or CXP4087, as sold by Chris-Craft Industrial Products of Gary, Ind., US. Examples also include the materials M8630 and/or CXP4087 themselves. Other example PVA films are also available as “Solublon PT30” and “Solublon KA40” from Aicello Chemical Co., Ltd., Aichi, Japan.

Plasticizers can include water glycerol, ethylene glycol, diethyleneglycol, propylene glycol, sorbitol, and mixtures thereof. Other additives can be stabilizers, disintegrating aids, etc.

The at least one sachet can be made of a material which is stretchable, as set out herein. This facilitates the closure of the open sachet, when it is filled over than 90% or even 95% by volume or even 100% or even over-filled. The material is preferably elastic, to ensure tight packing and fixation of the nutrient concentrate therein during handling, e.g., to ensure no (additional) head space can be formed after closure of the at least one sachet. Preferred stretchable materials have a maximum stretching degree of at least 150%, at least 200%, or at least 400% as determined by comparison of the original length of a piece of material just prior to rupture due to stretching, when a force of from about 1 to about 20 Newtons is applied to a piece of film with a width of 1 cm. Preferably, the material is such that it has a stretching degree as before, when a force of from about 2 to about 12 Newtons, or about 3 to about 8 Newtons, is used. For example, a piece of film with a length of 10 cm and a width of 1 cm and a thickness of 40 pm is stretched lengthwise with an increasing stress, up to the point that it ruptures. The extent of elongation just before rupture can be determined by continuously measuring the length and the degree of stretching can be calculated. For example, a piece of film with an original length of 10 cm that is stretched with a force of 9.2 Newton to 52 cm just before breaking, has a maximum stretching degree of 520%.

The force to stretch such a piece of film (10 cm×1 cm×40 microns) to a degree of 200% can be within the ranges disclosed above. This can ensure that the elastic force remaining in the film after forming the at least one sachet or closing the at least one sachet is high enough to pack the nutrient concentrate tightly within the sachet (but not so high that the film cannot be drawn into a vacuum mold of reasonable depth, when the at least one sachet is made by a process involving the use of vacuum, such as by vacuum-forming or thermo-forming). The stretchable material is defined by a degree of stretching measured when it is not present as a closed sachet. However, the material can be stretched when forming or closing the sachet. This can for example been seen by printing a grid onto the material, e.g. film, prior to stretching, then forming a sachet; it can be seen that squares of the grid are elongated and thus stretched. The elasticity of the stretchable material can be defined as the “elasticity recovery”. This can be determined by stretching the material for example to an elongation of 200%, as set out above, and measuring the length of the material after release of the stretching force. For example, a piece of film of a length of 10 cm and width 1 cm and thickness of 40 pm is stretched lengthways to 20 cm (200% elongation) with a force of 2.8 Newtons (as above), and then the force is removed. The film snaps back to a length of 12 cm, which indicates an 80% elastic recovery. The sachet material can have an elasticity recovery of from about 20% to about 100%, about 50% to about 100%, about 60% to about 100%, about 75% to about 100%, or about 80% to about 100%.

The degree of stretching can be non-uniform over the at least one sachet, due to the formation and closing process. For example, when a film is positioned in a mold and an open sachet is formed by vacuum forming, the part of the film in the bottom of the mold, furthest removed form the points of closing, may be stretched more than in the top part. A stretching action, when using stretchable, elastic, or both, material stretches the material non-uniformly resulting in a sachet which has a non-uniform thickness. This may allow control of the dissolution/disintegration or dispersion of the sachets in the water added to the medium bag. The material can be stretched such that the thickness variation in the sachet formed of the stretched material is from 10 to 1000%, 20% to 600%, 40% to 500%, or 60% to 400%. This can be measured by any method, for example by use of an appropriate micrometer.

Similarly, the water-soluble film sheets can be of varying opacity such that one sheet may be at least substantially transparent while the other sheet may be opaque.

The at least one sachet may comprise a main compartment and at least one sub-compartment. The main compartment and sub-compartments may contain nutrient concentrates or optional additives. Alternatively, more than one sachet may be used, where instead of having a single sachet containing both nutrient concentrate and optional additives or other components (whether combined or in separate compartments within the same sachet), more than one sachet can be used where the nutrient concentrate and optional additives or other components are contained in separate sachets.

Optional additives include, but are not limited to, indicator compounds, dyes, quenchers and fixatives. Other additives may be reagents for the extraction and/or detection of organisms. Such reagents can be pure, formulated with additives, diluents or in combination with phages (submicroscopic, usually viral organisms that destroy bacteria), components derived from phages, antibodies, polyhistamines or maltose-binding domains or any other affinity peptides, aptomers, biotins or streptoavidins or any other affinity molecules. The formulated reagents can be in the form of a liquid, gel, paste, dry powder, granule, or other free-flowable form. Alternatively, the reagents can be immobilized on solid supports such as particles, particularly magnetic or paramagnetic particles of micron to nanometer sizes.

Preferably, the water-reactive sachet, matrix, coating or seal begins releasing the nutrient concentrate and/or any other additives and components almost immediately upon contacting water during sample preparation. For example, the sachet begins releasing its contents from about 1 second to about 120 seconds, or about 5 seconds to about 60 seconds, after contacting the water. The amount of water to which the at least one sachet can be added varies according to the type of complex sample mixture desired to be formed. Those skilled in the art recognize that such information is known in the art and readily available.

The at least one sachet can be used by itself where it can be added to a container, preferably a sterilized container, to form a complex sample mixture. Alternatively, the at least one sachet can be used in conjunction with a homogenizer bag. The homogenizer bag may itself have a main containment area and additional sub-containment area, where the sachet can be contained in either containment area until its time of use. Alternatively, the at least one sachet can be in a fixed position where it is integral with the homogenizer bag.

The at least one sachet is desirably prepared in a manner to provide a hermetic seal completely around the perimeter to fully enclose the interior of the sachet and its contents prior to time of use. A complete perimeter seal may maintain the interior of the sachet in a sterile condition. The at least one sachet may be cut or torn open below the top perimeter seal at the time of use to introduce the test sample into the homogenizer bag containing the water needed to constitute the medium, however a sachet comprising a water reactive material is preferred. Optionally, the at least one sachet can comprise a guiding means for facilitating the opening of the sachet. Such guiding means comprises at least one notch, perforation or a combination thereof incorporated in the sachet.

The at least one sachet can be partially assembled before introduction of the nutrient concentrate (i.e. as many operations in a sachet as possible are accomplished prior to introduction of the nutrient concentrate). For example, it may be desirable to assemble “blank” sachets. Partial assembly can produce nonspecific blank sachets that can be customized with different test-specific nutrient concentrate and optional additive packages.

The one or more sub-compartments, if present in a sachet, can be installed either during or after sachet formation. For example, the at least one sub-compartment can be formed by heat-sealing the overlying sheets at temperatures lower than that required to provide the lock-up perimeter seal. A frangible seal may run between two points on the perimeter seal such that the frangible seal and the portion of the perimeter seal between the points defines a separated compartment. A portion of the perimeter seal is left unsealed to provide an opening to introduce the nutrient concentrates. After the nutrient concentrate is introduced into the separated compartment, the opening in the perimeter seal is sealed to provide a closed compartment.

Sterilization of the at least one sachet and its contents occurs in a clean room under stringent conditions. The sterilized concentrate or other contents can be placed in the at least one sachet, either into a separated compartment or contained within a sachet, as disclosed above. The at least one sachet can be sterilized by means known to one skilled in the art such as in an autoclave at a temperature of 121° C., a pressure of 15 psi, and 100% steam. The sachets may also be sterilized by irradiation, a conventional procedure that is familiar to a skilled person. A non-sterile nutrient concentrate or other contents can be introduced into a non-sterile sachet and then both items are subjected to irradiation treatment as a single unit. Gamma rays or electron radiation may also render the unit sterile. It is preferred that any contaminants not be allowed to grow to significant levels prior to radiation treatment unless the radiation dosage is increased to completely kill these contaminants and may render one or more nutrients in the medium incapable of supporting the growth of desired organisms when subsequently used for culturing. Excessive growth of these contaminants prior to sterilization may result in the creation and accumulation of toxic waste products that cannot be removed by sterilization, but may nevertheless restrict or prevent the growth of organisms during culturing. Control of the pre-sterilization growth of contaminants can include sterilizing within a short period of time after filling (e.g., 48 hours) or by refrigerating to restrict the growth of the contaminants. Radiation dosage required for sterilization is well known to one skilled in the art and may depend on sachet material and type of medium. For example, a gamma radiation dose of 2.5 Mrads may be sufficient to kill contaminants. Gamma radiation in the range of 15 to 30 kGy can be used.

The nutrient concentrate can comprise a pellet, granule, powder, sheet, plaque or the like. Suitable nutrients are well known to those skilled in the art. Similarly, the amounts of nutrient concentrate needed to form the appropriate nutrient medium is also well-known to those skilled in the art. The nutrient concentrate can be contained in either a single sachet or multiple sachets. Additionally, the nutrient concentrate components can be contained in separate sachets and come together upon addition to water.

The nutrient concentrate comprises various nutrients suitable for supporting the growth of organisms (e.g. proteins, amino acids, vitamins, sugars, oxygen and the like). The nutrient concentrate may further comprise components such as inorganic salts, buffers, indicators and the like to facilitate sample culturing and analysis. Preparations of nutrients are known in the art and are available in concentrated form derived from, for example, soybean casein, thioglycolate and brain and/or heart infusions. The nutrient concentrate is typically a solid, and may be in flowable forms such as dusts powders, granules and the like. Alternatively, the concentrate may be compressed into pellets sheets, plaques or the like, with or without any additional binders. Semisolid, paste, gel or concentrated liquid forms may also be suitable for use in some embodiments of the invention.

Addition of the food sample to the container, preferably a sterilized homogenizer bag is well known in the art.

With respect to target organism amplification, in order to identify a target by the present method, bacterial cells contained within a complex sample mixture and grown in a non-selective media are lysed, for example in a lysis buffer, to release total target organism nucleic acid. Target nucleic acid is then amplified according to a standard method for primer directed amplification. Typically, PCR is used and follows a standard thermocycling procedure in the presence of an appropriate nucleic acid replication composition. A suitable nucleic acid replication composition will contain, for example, dATP, dCTP, dGTP, dTTP, target specific primers and a suitable polymerase. Primers will be selected to specifically amplify target nucleic acid. If nucleic acid composition is in liquid form, suitable buffers known in the art are used. (Sambrook, J. et al., Molecular Cloning: A Laboratory Manual, Second Edition, Cold Spring Harbor Laboratory Press (1989)).

Target nucleic acid is any nucleic acid that is specific to, and may be used to identify a particular target organism. A large number of sequences have been identified that are specific to various organisms, such as pathogenic bacteria, including, but not limited to, those set forth below, which may serve as the target sequence in the invention, depending on the organism to be detected. Use of the appropriate target sequence is known to those skilled in the art. For the detection of pathogenic E. coli for example, Samapour (J. Clin. Microbiol. (1995), 33(8), 2150-4) teaches the detection of E. coli 0157:H7 by restriction fragment length polymorphism using Shiga-like toxin genes which are conserved between the 0157:H7 serotype and shigella. Similarly, Ramotar et al. (J. Clin. Microbiol. (1995), 33(3), 519-24) and Fratamico et al. (J. Clin. Microbiol. (1995), 33(8), 2188-91) teach PCR based methods for the detection of conserved 0157:H7 genes encoding either shiga-like toxins or verotoxins. Similar sequence have been identified for the detection of Listeria. In U.S. Pat. No. 5,523,205 and JP 05219997 DNA probes capable of hybridizing to a portion of the genome of pathogenic Listeria monocytogenes, are disclosed. DE 4238699 and EP 576842 teach methods for detection of Listeria monocytogenes using primers designed to give amplification products specific to the monocytogenes genome and EP 576842 describes amplification primers based on genes encoding the highly conserved iap (invasion-associated protein) of Listeria. Finally, WO 9500664; WO 9425597 and WO 9425595 all disclose sequences derived from the Salmonella genome useful for the specific detection and identification of Salmonella species.

A control nucleic acid fragment is preferably amplified concurrently with the target nucleic acid. The control nucleic acid fragment may be designed to be amplified either with a single primer that is identical to one of the primers used in the amplification of the target genomic nucleic acid or with different primers. The control nucleic acid fragment is useful to validate the amplification reaction. Amplification of the control nucleic acid fragment may be accomplished concurrently with the test sample containing the target nucleic acid. If the control shows amplification, there is positive indication that the procedure has been effective regardless of the positive or negative results attained in the concurrent test. In order to achieve significant validation of the amplification reaction a suitable number of copies of the control nucleic acid fragment must be included in each amplification reaction. Copies of control nucleic acid fragment per reaction may range from 10 copies to 1×10⁴ copies where 100 copies to 1000 copies are preferred. The control nucleic acid fragment may be constructed according to those methods known in the art. Control nucleic acid fragment will be of appropriate size and base composition to permit amplification by a method primer directed amplification. The control nucleic acid fragment may be isolated from the target organism, or from another source, but must be reproducibly amplified under the same conditions that permit the amplification of the target nucleic acid. In a preferred embodiment, the control nucleic acid is similar in size and base composition to the target nucleic acid to be detected.

Alternatively if the composition is contained in a tabletted reagent, then typical tabletting reagents are included such as stabilizers and the like. Within the context of the invention replication compositions will be modified depending on whether they are designed to be used to amplify target nucleic acid or the control nucleic acid fragment. Replication compositions that will amplify the target nucleic acid (test replication compositions) will generally include (i) a polymerase (generally thermostable), (ii) a primer pair capable of hybridizing to the target nucleic acid and (iii) buffers for the amplification reaction to proceed. Replication compositions that will amplify the control nucleic acid (positive control, or positive replication composition) will generally include (i) a polymerase (generally thermostable) (ii) the control nucleic acid fragment; (iii) at least one primer capable of hybridizing to the control nucleic acid fragment; and (iv) buffers for the amplification reaction to proceed. In some instances it may be useful to include a negative control replication composition. The negative control composition will contain the same reagents as the test composition but without the polymerase. The primary function of such a control is to monitor spurious background fluorescence in a homogeneous format when the method employs a fluorescent means of detection.

Replication compositions may be in either liquid or tabletted form, where a tablet is preferred for ease of use. Tablets are prepared according to the “snow gun” process, fully described in U.S. Pat. No. 5,307,640 (Fawzey et al.); U.S. Pat. No. 4,762,857 (Bollin, Jr. et al.); U.S. Pat. No. 4,678,812 (Bollin, Jr. et al.), U.S. Pat. No. 3,932,943 (Briggs et al.), and U.S. Pat. No. 5,475,984 (application Ser. No. 08/298,231) (Fermani et al.). In general, the control and test compositions are frozen into particles by means of a cryogenic liquid, the particles providing feedstock for tabletting. The control and test compositions may be formed into separate tablets or combined in a single tablet.

The snow gun process uses a cryogenic liquid for producing frozen particles of a liquid product in a housing which comprises the steps of: (a) introducing the cryogenic liquid into the housing in an annular, downward direction creating a substantially continuous downwardly directed circumferential wall of cryogenic liquid, defining an interior entrapment zone; and (b) introducing droplets of the liquid product into the entrapment zone, whereby the cryogenic liquid freezes the liquid product droplets to produce frozen particles.

Detection of the amplified target nucleic acid and the control may be accomplished by any methods known in the art, including by gel electrophoresis or by fluorescent detection methods. The latter method is particularly useful when carried out in a homogeneous format, for example, where fluorescence emissions from dyes, incorporated in the amplification products, may be detected without the separation of products from primers or nucleic acid templates.

Methods of gel electrophoresis of nucleic acids are common and well known in the art, and may be practiced according to a variety of protocols including those found in Southern, E. M et al., Pulsed Field Gel Electrophoresis. (1995), 1-19. Editor(s): Monaco, Anthony P. Publisher: IRL Press, Oxford, UK.

Where fluorescence detection is used, a fluorescent intercalating dye is employed to detect the presence of amplification products. The intercalating dye, as described below, may be added either before or after nucleic acid amplification, depending on the properties of the dye. Excitation of control or test samples containing amplification products will result in a specific wave length emitted. Measurement and comparison of light emission from the control and test samples provide a means of determining the presence of amplification products.

Where a fluorescent means of detection of nucleic acid amplification products is used, an intercalating agent capable of binding to double stranded DNA (dsDNA) and emitting a fluorescent signal is a preferred reagent. A variety of suitable intercalating agents are known in the art such as propidium iodide (PI) and ethidium bromide (EB) [Sailer et al., Cytometry (1996), 25(2), 164-172] Oxazole Yellow [EP 714986], TO-TO™ (1,1′-(4,4,7,7-tetramethyl-4,7-diazaundecamethylene)-bis-4-[3-methyl-2,3-dihydro-(benzo-1,3-thiazole)-2-methylidene]-quinolinium tetraiodide), a homodimer of thiazole orange [Axton et al., Mol. Cell. Probes (1994), 8(3), 245-50] oxazole orange (YOYO) [Srinivasan et al., Appl. Theor. Electrophor. (1993), 3(5), 235-9] as well as the cyanine dyes [U.S. Pat. No. 5,563,037]. Preferred in the present method are the unsymmetrical cyanine dyes such as are discussed in U.S. Pat. No. 5,563,037; U.S. Pat. No. 5,534,416; U.S. Pat. No. 5,321,130 and U.S. Pat. No. 5,436,134 hereby incorporated by reference.

Where it is preferred that the intercalating dye be added during or before the nucleic acid amplification reaction, a dye must be chosen that is both thermostable and will not inhibit the amplification reaction. Most suitable are the cyanine dyes YO-PRO-1™ (Quinolinium,4-[(3-methyl-2(3H)-benzoxazolylidene)methyl]-1-[3(trimethylammonio)propyl]-,diiodide) and SYBR GREEN® available from Molecular Probes, Inc. (Eugene, Oreg.). These dyes are particularly suited for use in the invention due to their high extinction coefficient, near zero fluorescence when unbound to DNA, suitable binding affinity to double-stranded DNA and reasonable photostability. Further, both dyes are sufficiently resistant to the elevated processing temperatures at the time intervals used to provide an effective signal during the amplification reaction. Cyanine dyes which are particularly suited for use prior to or during DNA amplification generally will have binding constants from about from about 1×10⁴ to about 5×10⁵ (molar⁻¹).

Where interference with thermocyling is not an issue it is possible to expand the list of suitable intercalating agents to include those with binding constants higher than 5×10⁵ (molar.sup.−1). Intercalating agents with binding constants at this level are expected to interfere with the primer directed amplification and thus are not good candidates for addition to an amplification reaction during or prior to thermocycling. For example TO-TO-1™Quinolinium, 1,1′-[1,3-propanediylbis[(dimethyliminio)-3,1-propanediyl]]bis[4-[(3-methy1-2(3H)-benzothiazolylidene)methyl]]-,tetraiodide, will interfere with the DNA amplification reaction, but if added to the sample after amplification, it is a very serviceable fluorescent indicator.

As mentioned, the intercalating agent can be provided at any step of the method prior to fluorescence detection. For example, the intercalating agent may be present in either the test or control replication composition, may be added during thermocyling or may be added just prior to fluorescence detection. Typically the dye is added to the sample to give a final concentration of about 3 uM. Thermocycling proceeds according to typical cycling times and temperatures.

The intercalating agent chosen for use in the instant method may be temperature sensitive; i.e., the binding affinity of the intercalating agent for dsDNA and hence the magnitude of the fluorescent signal emitted may vary with temperature. Accordingly, it is readily apparent to one skilled in the art that instrument calibration, positive and negative controls and samples must all be assayed under controlled temperature conditions. Alternatively, a mathematical algorithm may be developed in order to compensate for variations in ambient and calibration temperatures. For example, the following algorithm comprises a simple linear multiplier that calculates the fluorescence value at a standard calibration temperature (FlUc) as a function of the ambient temperature at which sample measurements are taken (t) and the fluorescence intensity units (FfUt) recorded at that temperature:
FIUc=FIUtX(TCF), wherein

FIUc=the calculated fluorescence value;

FIUt=the measured fluorescence value at a given ambient temperature (t); and
TCF=((0.25+0.05(t))/1.45.

This algorithm produces a constant result over a temperature range of 15-35° C.

Alternatively, homogenous detection may be employed to carry out “real-time” primer-directed nucleic acid amplifications, using primer pairs of the instant invention (e.g., “real-time” PCR and “real-time” RT-PCR). Preferred “real-time” methods are set forth, for example, in U.S. Pat. Nos. 6,171,785 and 5,994,056, each of which is hereby incorporated by reference in its entirety.

Another detection method that may be employed is the 5′ nuclease detection method, as set forth, for example, in U.S. Pat. Nos. 5,804,375, 5,538,848, 5,487,972, and 5,210,015, each of which is hereby incorporated by reference in its entirety.

What is claimed is:

1. A method of detecting a target organism in a sample mixture comprising:
(a) preparing at least one complex sample mixture, said preparing step comprising adding to a container:
(i) at least one sachet containing at least a nutrient concentrate; and
(ii) a sample mixture suspected of containing a target organism;
(b) performing primer directed nucleic acid amplification of the at least one complex sample mixture; and
(c) examining the primer directed nucleic acid amplification result of step (b) to detect for presence or absence of amplification product of the target organism, wherein the presence or absence of the amplification product is indicative of the presence or absence of the target organism in the sample mixture.

2. The method according to claim 1, wherein the sample mixture is a food sample mixture.

3. The method according to claim 1, wherein the primer directed nucleic acid amplification is polymerase chain reaction amplification.

4. The method according to claim 1, wherein the target organism is a pathogenic bacteria.

5. The method according to claim 1, wherein the target organism is selected from the group consisting of Salmonella, Listeria, E. coli, Campylobacter, Enterobacter sakazakii, Vibrio, and Clostridia.

6. The method according to claim 1, wherein the at least one sachet comprises a water-reactive material, wherein the water-reactive material dissolves, ruptures, disperses and/or disintegrates upon contact with water.

7. The method according to claim 6, wherein the water-reactive material is water-soluble.

8. The method according to claim 7, wherein the at least one sachet has a solubility in water of at least 50%.

9. The method according to claim 1, wherein the at least one sachet comprises two overlaid sheets of water-soluble film that are sealed together.

10. The method according to claim 1, wherein the at least one sachet comprises a film of at least one polymeric material.

11. The method according to claim 10, wherein the at least one polymeric material comprises at least one of polyvinyl alcohols, polyvinyl pyrrolidone, polyalkylene oxides, polyacrylamide, polyacrylic acid, cellulose, cellulose ethers, cellulose esters, cellulose amides, polyvinyl acetates, polycarboxylic acids and salts, polyaminoacids or peptides, polyamides, polyacrylamide, copolymers of maleic/acrylic acids, polysaccharides, and natural gums.

12. The method according to claim 1, wherein the at least one sachet comprises a main compartment and at least one sub-compartment.

13. The method according to claim 1, wherein the container is a sterilized container.

14. The method according to claim 1, wherein the container is a homogenizer bag.

15. The method according to claim 1, wherein the at least one sachet is hermetically sealed.

16. The method according to claim 1, wherein the nutrient concentrate comprises a pellet, granule, powder, sheet, or plaque.

17. The method according to claim 1, wherein the nutrient concentrate comprises at least one of proteins, amino acids, vitamins, sugars, oxygen, soybean casein, thioglycolate, brain and heart infusions.

18. The method according to claim 17, wherein the nutrient concentrate further comprises inorganic salts, buffers and indicators.
